(19) United States
(12) Patent Application Publication
(10) Pub. No.: US 2024/0247252 A1
(43) Pub. Date: Jul. 25, 2024

(54) NOVEL COMPOUNDS FOR THE DEVELOPMENT OF REVERSIBLE COVALENT DRUGS

(71) Applicant: The Trustees of Boston College, Chestnut Hill, MA (US)

(72) Inventor: Jianmin Gao, Newton, MA (US)

(73) Assignee: The Trustees of Boston College, Chestnut Hill, MA (US)

(21) Appl. No.: 18/405,421

(22) Filed: Jan. 5, 2024

Related U.S. Application Data
(60) Provisional application No. 63/478,618, filed on Jan. 5, 2023.

(51) Int. Cl. C12N 15/10; C07F 5/02; C07K 7/50

(52) U.S. Cl. CPC C12N 15/1037; C07F 5/025; C07K 7/50

(57) ABSTRACT

This patent document provides novel compounds as warheads for therapeutic or diagnostic agents. Advantages of the agents modified with the warheads include low off-target conjugation and minimized immunogenicity due to the reversible covalent bonding between the warheads and the targets.

### CROSS-REFERENCE TO RELATED APPLICATIONS

This application claims priority to Provisional Application No. 63/478,618, filed Jan. 5, 2023, the disclosures of which is hereby incorporated by reference in the entirety.

### STATEMENT REGARDING FEDERALLY SPONSORED RESEARCH

This invention was made with government support under GM102735 awarded by the National Institutes of Health and under CHE1904874 awarded by the National Science Foundation. The government has certain rights in the invention.

### TECHNICAL FIELD

The present invention relates to novel compounds as warheads for therapeutic or diagnostic agents. The agents modified with the warheads benefit from reversible covalent bonding between the warheads and the targets.

### BACKGROUND

Targeted covalent inhibitors (TCIs) represent a class of small molecules designed to selectively bind to specific target proteins, forming covalent bonds. This approach contrasts with traditional non-covalent inhibitors, which form reversible interactions with their target proteins. TCIs have gained significant attention in the field of drug discovery and development due to their potential advantages, such as enhanced selectivity, prolonged pharmacological effects, and the ability to target proteins with challenging binding sites. Typically, a TCI consists of a reactive functional group (warhead) as part of a larger molecular scaffold. The molecular scaffold is expected to bind the target protein noncovalently, positioning the warhead to react with a protein residue and achieve covalent inhibition. Not surprisingly, given the mechanism of action, the warhead and its carrier scaffold will have to be carefully matched and fused into the TCI with atomic level prevision, which presents a major challenge to TCI discovery.

There is an ongoing need in the art to develop new warheads of TCIs for maintaining therapeutic efficacies and meanwhile reducing off-target conjugation and immunogenicity.

### SUMMARY

The compounds of this patent document address the need. Novel warheads that can form reversible covalent bonds have been developed. Incorporating such reversible covalent warheads into TCIs give rise to reversible covalent drugs, which in comparison to their irreversible counterparts may enjoy the benefits of low off-target conjugation and minimized immunogenicity.

The incorporation of one or more oxalimidic acids in one single molecule can provide interaction sites/adhesion points to the crystal surface thus providing strong affinity to the crystal surface and prevent the attachment of additional calcium oxalates from binding to the crystal surface hence inhibit crystal growth.

An aspect of this patent document provides a compound represented by Formula I,

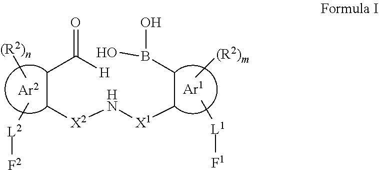

### Wherein:

Ar¹ and Ar² are each independently a 5-10 membered aromatic ring,
X¹ is C₁₋₂ alkylene or CH═CH (optionally substituted with for example C₁₋₆alkyl), X¹ is in a para position to the boron moiety of Ar¹,
X² is void or CH₂, X¹ is in a para position to the CHO moiety of Ar²,
L¹ and L² are each independently void or a linker, provided that L¹ and L² are not void at the same time,
F¹ and F² are each independently void or a functional group capable of reacting with OH, SH or amino group to form a covalent bond,
R¹ and R² in each instance are independently selected from the group consisting of C₁₋₆alkyl, OC₁₋₆alkyl, C₁₋₄alkyl-C(O), CN, NO₂, OH, COOR, halogen, C₁₋₄alkyl-O, N(R^{a})₂, C₁₋₆alkyl-O—C(O), C₁₋₆alkyl-C(O)—O, C₁₋₆alkyl-C(O)—NR^{a}, C₁₋₆alkyl-NR^{a}—C(O), wherein R^{a} in each instance is independently H or C₁₋₆alkyl,
m and n are each independently 0, 1, 2, 3, 4 or 5.

Another aspect of this patent document provides a peptide comprising a residue modified by the compound disclosed herein, wherein the peptide is represented by Formula II,

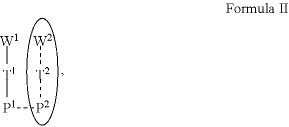

wherein the circled W²-T²-P² is optional, W¹ and W² are each independently derived from Formula I, T¹ and T² are each independently a di-functional or tri-functional linkage, P¹ and P² are each independently a peptide comprising three or more amino acid residues.

Another aspect of this disclosure provides a phage display library comprising phage particles comprising a peptide disclosed herein.

Another aspect provides a method of identifying a targeting peptide that binds to a target of interest, comprising:
a) providing a plurality of candidate peptides disclosed herein;
b) contacting the candidate peptides with the target of interest; and
c) selecting from the candidate peptides a peptide having an affinity to the target of interest that is submicromolar, thereby identifying said targeting peptide.

In some embodiments, the method includes conjugating the selected targeting peptide with an agent. In some embodiments, each candidate peptide is attached to a bacteriophage coat protein.

Another aspect provides a method of developing a novel therapeutic agent against a target of interest. The method includes conjugating the targeting peptide identified according to the method of disclosed herein with a therapeutic agent against the target of interest. In some embodiments, the target of interest is a bacteria of Staphylococcus aureus or Acinetobacter baumannii strain.

### DESCRIPTIONS OF THE DRAWINGS

FIG. 1 illustrates reversible covalent inhibition of SrtA. FIG. 1(a) shows SrtA structure with docked W7 (PDB: 1t2w). The peptide's termini and the proximal lysine residues are labeled with the lysine ε-amines colored blue. K173 (in light blue shade) was found to be the conjugation site for P5. FIG. 1 (b) shows Chemical structure of W7 and the warheads for its modification. FIG. 1(c) shows IC₅₀ values of various peptides measured through competition against W7-F. C^{#} represent iodoacetamide-alkylated cysteines. ^{¥}P5 was N-acetylated to prevent RMR1 conjugation with the N-terminus. FIG. 1(d) and FIG. 1(e) show SrtA binding kinetics of P3 and P5 recorded via a competition assay using W7-F as a fluorescence reporter.

### DETAILED DESCRIPTION

Various embodiments of this patent document disclose a novel class of compounds that serve as warheads for reversible covalent therapeutic agents (e.g. inhibitors) and enhance binding and inhibition as a result of their covalent bonding to the target. These compounds also allow for low off-rate to maximize the thermodynamic benefit of the warhead and achieve long-lasting inhibition. The development of the new warheads taps into the power of boron chemistry. Boron is largely absent from native biomolecules; however, its versatile reactivity has been long recognized in synthetic organic chemistry. On the other hand, although foreign to native biomolecules, boron can be well tolerated by biological systems as seen with the success of organoboron based therapeutics.

While the following text may reference or exemplify specific embodiments of a compound or a method of treating a disease or condition, it is not intended to limit the scope of the compound or method to such particular reference or examples. Various modifications may be made by those skilled in the art, in view of practical and economic considerations, such as the substitutions of the compound and the amount or administration of the compound for treating or preventing a disease or condition.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, and topical administration. In some embodiments, pharmaceutically acceptable salts of the compounds disclosed herein are provided.

The term “subject” encompasses any animal, but preferably a mammal, e.g., human, non-human primate, a dog, a cat, a horse, a cow, or a rodent. More preferably, the subject is a human.

The term “carrier” refers to a chemical compound that facilitates the incorporation of a compound into cells or tissues.

The term “physiologically acceptable” or “pharmaceutically acceptable” refers to a carrier or diluent that does not abrogate the biological activity and properties of the compound.

The term “therapeutically effective amount” refers to an amount of a compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The term “alkyl” refers to monovalent saturated alkane radical groups particularly having up to about 18 carbon atoms, more particularly as a lower alkyl, from 1 to 8 carbon atoms and still more particularly, from 1 to 6 carbon atoms. The hydrocarbon chain may be either straight-chained or branched. The term “C₁₋₁₀ alkyl” or “C₁-C₁₀ alkyl” refers to alkyl groups having 1, 2, 3, 4, 5, 6, 7, 8, 9 or 10 carbon atoms. Similarly, the term “C₁₋₄alkyl” refers to alkyl groups having 1, 2, 3, or 4 carbon atoms. Non-limiting examples of alkyls include groups such as methyl, ethyl, n-propyl, isopropyl, n-butyl, iso-butyl, tert-butyl, n-hexyl, n-octyl, tert-octyl and the like.

The term “alkylene” refers to a divalent hydrocarbon which may be either straight-chained or branched. Different from alkyl which has only one point of bonding with other groups or atoms, alkylene has two points of bonding. Non-limiting examples include groups such as CH₂, (CH₂)₂, CH₂CH(CH₃), and the like. A C₁₋₈alkylene has 1, 2, 3, 4, 5, 6, 7 or 8 carbons. A C₁₋₄ alkylene has 1, 2, 3 or 4 carbons.

The term “alkenyl” refers to a monovalent hydrocarbon containing at least a double bond between two adjacent carbons. The term “alkenylene” refers to divalent hydrocarbon containing at least a double bond between two adjacent carbons. Nonlimiting examples of “alkenylene” include ethenylene (CH═CH) and propenylene.

The term “C₁₋₄ alkoxy” includes an alkyoxy group having 1, 2, 3 or 4 carbons.

The term “carbocycle” or “cycloalkyl” refers to 3 to 10 membered cyclic hydrocarbyl groups having only carbon atoms as ring atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

The term “heterocycle” or “heterocycloalkyl” refers to 3 to 10 membered substituted or nonsubstituted non-armoatic cyclic groups where one or more carbon ring atoms are replaced with hetero atoms or groups containing heteroatoms (e.g. NH, NCl—C₄alkyl O, and S). Nonlimiting examples include pyrrolidine, piperidine, N-methyl-piperizine, and morpholine. Optional substituents include C₁₋₆ alkyl, C₁₋₄ alkoxy, halogen, haloalkyl, sulfonamido, and amido.

The term “carbo-aryl” or “aryl” is intended to mean any stable monocyclic or bicyclic carbon ring of up to 7 members in each ring, wherein at least one ring is aromatic and all ring atoms of the aromatic ring are carbon atoms. Typical aryl groups include, but are not limited to, groups derived from aceanthrylene, acephenanthrylene, anthracene, azulene, benzene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, and the like. Particularly, an aryl group comprises from 6 to 10 or 6 to 14 carbon atoms.

The term “hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloheteroalkyl.

The term “halogen” refers to F, Cl, Br, or I.

The term “heteroaryl” refers to groups having 5 to 14 ring atoms, preferably 5, 6, 9, or 10 ring atoms, having 6, 10, or 14π electrons shared in a cyclic array, wherein at least one ring atom contributing to the shared π electrons in the cyclic array is a heteroatom. Typical heteroaryl groups include, but are not limited to, groups derived from acridine, carbazole, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, phenanthridine, phenanthroline, phenazine, phthalazine, phthalimide, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-15 membered heteroaryl, with 5-10 membered heteroaryl being particularly preferred.

An aspect of this patent document provides a compound or a pharmaceutically acceptable salt thereof, wherein the compound is represented by Formula I,

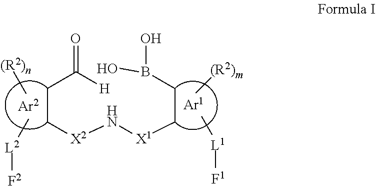

### Wherein:

Ar¹ and Ar² are each independently a 5-10 membered aromatic ring,
X¹ is C₁₋₂ alkylene or CH═CH (optionally substituted with for example C₁₋₆alkyl), X¹ is in a para position to the boron moiety of Ar¹,
X² is void or CH₂, X¹ is in a para position to the CHO moiety of Ar²,
L¹ and L² are each independently void or a linker, provided that L¹ and L² are not void at the same time,
F¹ and F² are each independently void or a functional group capable of reacting with OH, SH or amino group to form a covalent bond,
R¹ and R² in each instance are independently selected from the group consisting of C₁₋₆alkyl, OC₁₋₆alkyl, C₁₋₄alkyl-C(O), CN, NO₂, OH, COOR, halogen, C₁₋₄alkyl-O, N(R^{a})₂, C₁₋₆alkyl-O—C(O), C₁₋₆ alkyl-C(O)—O, C₁₋₆alkyl-C(O)—NR^{a}, C₁₋₆alkyl-NR^{a}—C(O), wherein R^{a} in each instance is independently H or C₁₋₆alkyl,
m and n are each independently 0, 1, 2, 3, 4 or 5.

The aromatic ring for Ar¹ and Ar² can be a carbo-aryl or a herteroaryl ring. Nonlimiting examples include the following optionally substituted ring: phenyl, pyridinyl, pyrimidyl, furanyl, thiophenyl, and pyrrolyl.

Additional examples for Ar¹ and Ar² include the following rings, which can be further substituted with one or more R¹ or R². Of course, the rings exemplified below for Ar¹ can also be used for Ar², and vice versa.

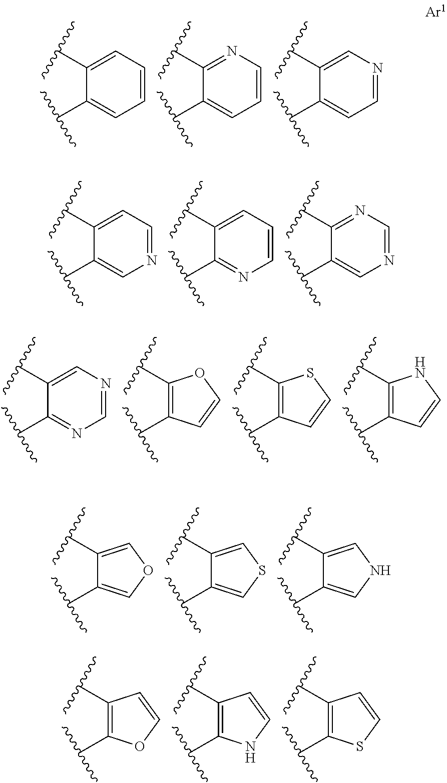

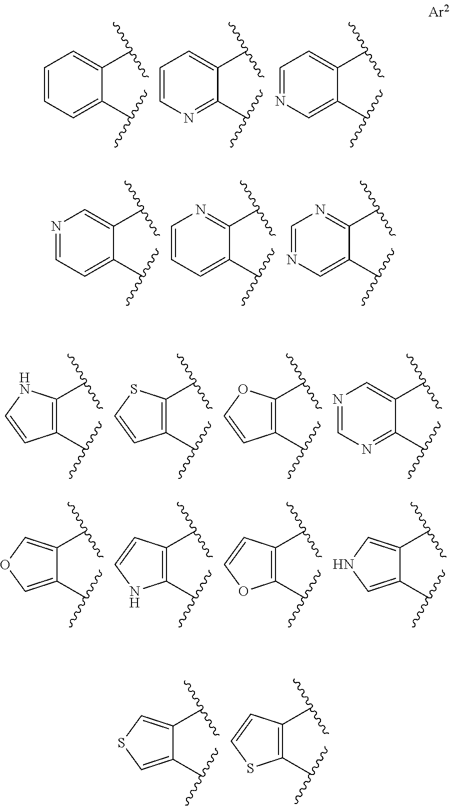

In some embodiments, Ar¹ and Ar² are each independently a phenyl or a heteroaryl. In some embodiments, one of Ar¹ and Ar² is a phenyl and the other is a heteroaryl. In some embodiments, both of Ar¹ and Ar² are each an optionally substituted phenyl.

In some embodiments, Ar¹ and Ar² are linked via X¹NHX² as the following. As descried above, each of Ar¹ and Ar² can be further substituted. The linkage with L¹ and L² may be an amide, an ester, an ether, a carbon-carbon bond, a sulfonamide, a sulfone, or any suitable form.

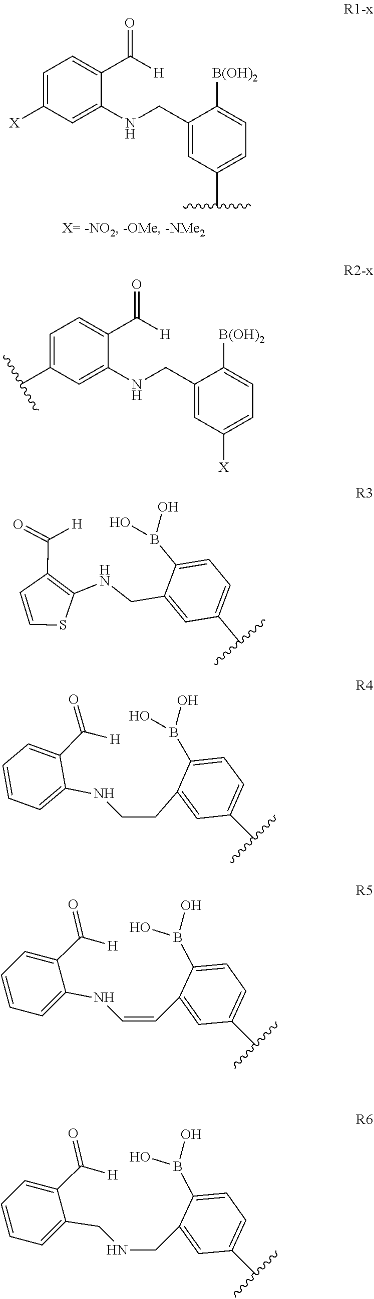

R¹ and R² at each substitutable carbon of Ar¹ and Ar² may be same or different. Nonlimiting examples of Rand R² include F, Cl, Br, I, COOH, OMe, OH, NO2, NH2, and CN.

L¹ and L² may derive or contain one or more components selected from, for example, C₁₋₈alkylene, NH—C₁₋₈alkylene, NH—C₁₋₈alkylene-NH, O, C═O, 5-8 membered heterocyclic, 6-10 membered carbo-aryl, —(CH₂)ₐC(O)NR^{a}(CH₂)_{b}—, —(CH₂)ₐO(CH₂CH₂O)_{c}—, —(CH₂)ₐheterocyclyl-, —(CH₂)ₐC(O)—, —(CH₂)ₐNR^{a}—, —CR^{a}═N—NR^{a}—, —CR^{a}═N—O—, —CR^{a}═N—NR^{b}—CO—, —N═N—CO—, —S—S—, and one or more natural or unnatural amino acid, wherein a, b, and c are each an integer selected from 0 to 25, all subunits included; and R^{a} and R^{b} in each instance independently represent hydrogen or a C₁-C₁₀ alkyl.

In some embodiments, one or both of L¹ and L² independently comprise one or more units selected from the group consisting of —C(O)NHC₂₋₈alkyleneNH—, —NHC₂₋₈alkyleneNH—, —NHC(O)C₁₋₆alkyleneNH—, —NHC(O)NHC₁₋₈alkyleneNH—, C₁₋₈alkylene, NH—C₁₋₈alkylene, amino acid, heterocyclic, —C₁₋₈alkyleneC(O)NR^{a}C₁₋₈alkylene-, —(CH₂)ₐO(CH₂CH₂O)_{c}—, —C₁₋₈alkyleneC(O)—, and —S—S—, wherein a, b, and c are each an integer selected from 0 to 25, all subunits included.

Nonlimiting examples of L¹ and L² include the following in connection with their respective Ar and F moieties. The structural components exemplified below for L¹ can also be used for L², and vice versa.

L¹ in connection with Ar¹ and F¹

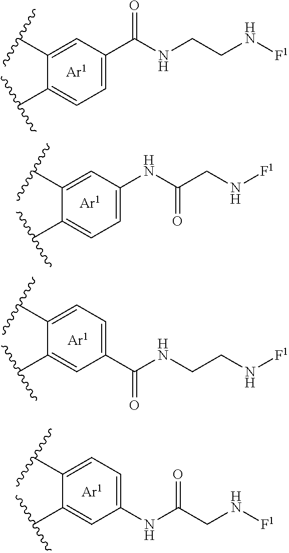

L² in connection with Ar² and F²

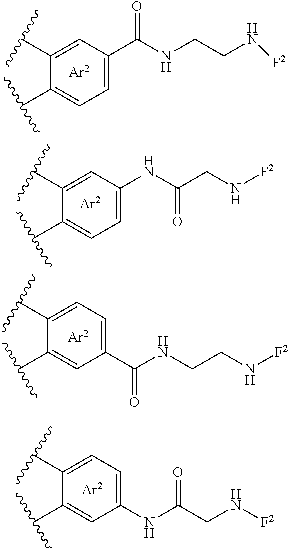

The functional group of F¹ and F² serve to form a linkage point with a therapeutic agent. Nonlimiting examples of F¹ and F² include amino, COOH, maleimide, 2′-pyridyldithio variant, bromo or iodo acetamide, α,β-unsaturated sulfonamide (e.g. CH₂═CHSO₂NHR, R=alkyl or aryl), aromatic or vinyl sulfone, α,β-unsaturated amide, α,β-unsaturated ester, azide, chloro-oxyme (e.g. HON═CClR, R=alkyl or aryl), alkyne, dibenzocyclooctyl (DBCO), carbonyl.

F¹ and F² may each have two or more functional groups for reacting with different moieties of a molecule (e.g. peptide). Nonlimiting examples of F¹ and F² (with or without a linker) include the following.

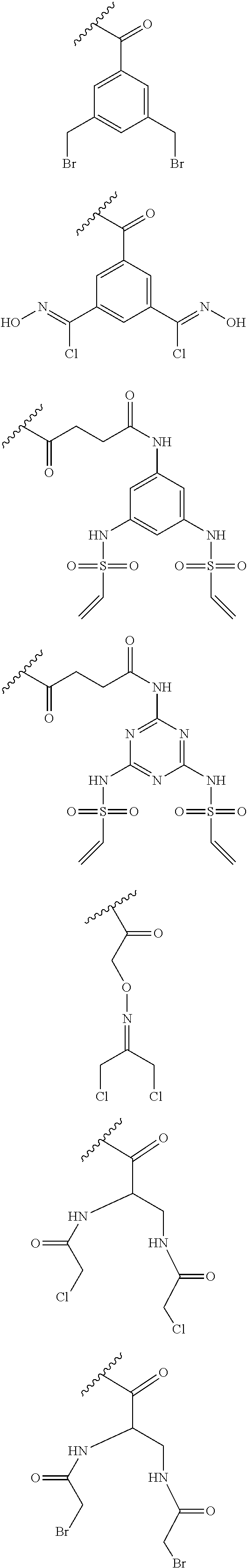

In some embodiments, at least one of F¹ and F² is COOH.

In some embodiments, X¹ is methylene. In some embodiments, X¹ is ethylene or ethenylene. In some embodiments, X² is void (NH connecting directly to Ar²).

Another aspect of the patent document provides a peptide comprising a residue modified by the compound of Formula I, wherein the peptide is represented by Formula II, wherein the circled W²-T²-P² is optional. W¹ and W² are each independently derived from Formula I, T¹ and T² are each independently a di-functional or tri-functional linkage, P¹ and P² are each independently a peptide comprising three or more amino acid residues. The scope of linkers, substituents, and functional groups also encompasses those described for Formula I.

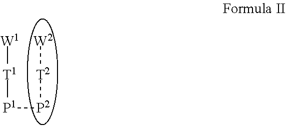

In some embodiments, T¹ is a bifunctional linkage. In some embodiments, T¹ is a trifunctional linkage. In some embodiments, T¹ is a trifunctional linkage linking W¹ with two amino acid residues.

In some embodiments, W²-T²-P² is present, and wherein W¹ and W² are different.

In some embodiments, W²-T²-P² is present and wherein T¹ and T² are linked to each other.

In some embodiments, the modified residue is a cysteine residue or a lysine residue

In some embodiments, P¹ and P² each independently comprise a peptide sequence represented by XR*(X)ₘ, wherein R* indicates the modified residue; wherein each instance of X is an amino acid independently selected from D, E, K, R, H, Y, N, Q, S, T, G, A, V, L, I, M, P, F, and W; wherein m is an integer selected from 1, 2, 3, 4, 5, 6, 7, 8, 9, and 10.

In some embodiments, P¹ and P² are both present and the peptide sequence is represented by XR*(X)ₘR′*(X)ₙ, wherein R* and R′* each represents a modified residue; wherein each instance of X is an amino acid independently selected from D, E, K, R, H, Y, N, Q, S, T, G, A, V, L, I, M, P, F, and W; wherein m is an integer selected from 5, 6, 7, 8, 9, and 10; wherein n is an integer selected from 1, 2, 3, 4, and 5. In some embodiments, the terminal X adjacent to R* is proline.

In some embodiments, the peptide comprises two modified cysteine residues resulting from two cysteines reacting with one of F¹ and F².

Nonlimiting examples of Formula I include the following:

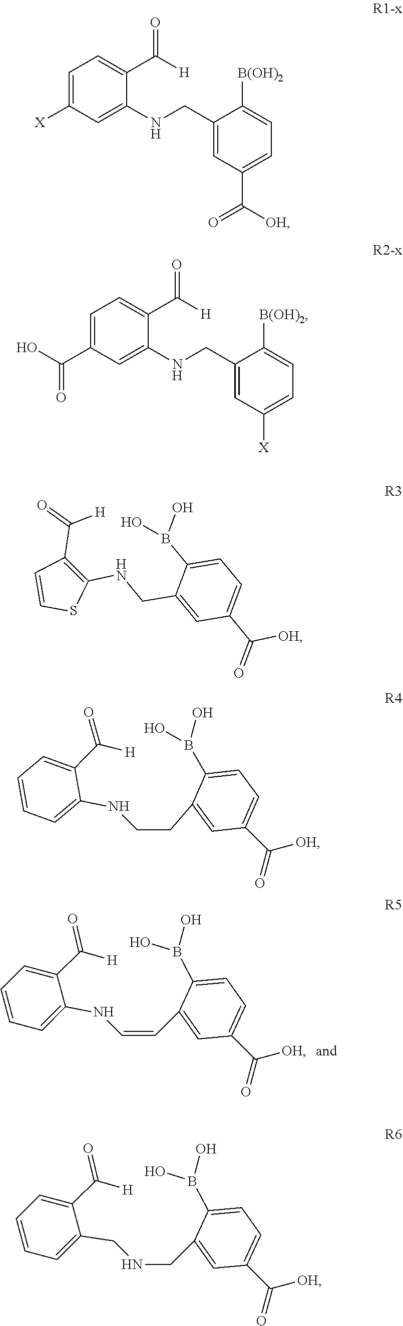

Wherein X is —NO₂, —OMe, or —NMe₂.

Additional examples of compounds of I include the following

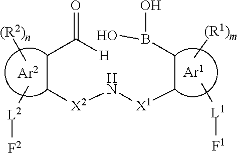

TABLE 1

| Com- | | | (R¹)ₘ or | | | | |
|---|---|---|---|---|---|---|---|
| pound | Ar¹ | Ar² | (R²)ₙ | L¹ - F¹ or F¹ | L² - F² or F² | X¹ | X² |
| 1 | Ph | Ph | F, Cl, Br, | COOH | void | CH₂ | void |
| 2 | Ph | Ph | F, Cl, methyl | 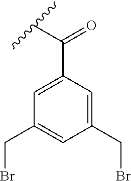 [SMILES: *C(=O)c1cc(CBr)cc(CBr)c1] | void | CH₂CH₂ | CH₂ |
| 3 | Ph | Ph | methyl, ethyl, Omethyl, | 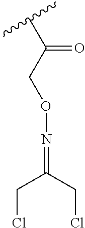 [SMILES: *C(=O)CON=C(CCl)CCl] | void | CH₂CH₂ | CH₂ |
| 4 | Ph | 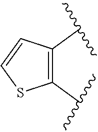 [SMILES: *c1ccsc1*] | Oethyl, NO₂, NMe₂, | 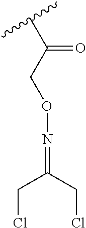 [SMILES: *C(=O)CON=C(CCl)CCl] | void | CHCH | void |
| 5 | Ph | 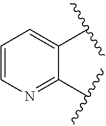 [SMILES: *c1cccnc1*] | F, Omethyl, NO₂, NMe₂, | 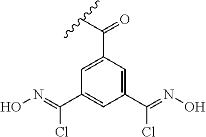 [SMILES: *C(=O)c1cc(/C(Cl)=N/O)cc(/C(Cl)=N/O)c1] | void | CHCH | void |
| 6 | 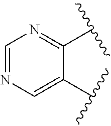 [SMILES: *c1cncnc1*] | Ph | Cl, methyl, ethyl, Omethyl, NMe₂, | void | 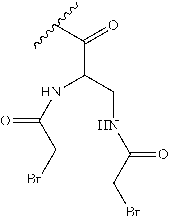 [SMILES: *C(=O)C(CNC(=O)CBr)NC(=O)CBr] | CH₂CH₂ | CH₂ |
| 7 | 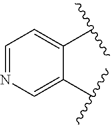 [SMILES: *c1ccncc1*] | Ph | methyl, NMe₂ | void | 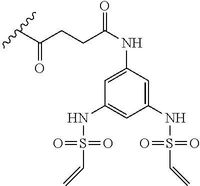 [SMILES: *C(=O)CCC(=O)Nc1cc(NS(=O)(=O)C=C)cc(NS(=O)(=O)C=C)c1] | CH₂ | CH₂ |
| 8 | Ph | 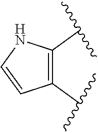 [SMILES: *c1cc[nH]c1*] | F, ethyl, NMe₂, | 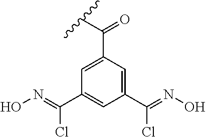 [SMILES: *C(=O)c1cc(/C(Cl)=N/O)cc(/C(Cl)=N/O)c1] | void | void | CHCH |
| 9 | Ph | Ph | F, ethyl, NMe₂, | 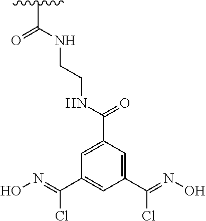 [SMILES: *C(=O)NCCNC(=O)c1cc(/C(Cl)=N/O)cc(/C(Cl)=N/O)c1] | void | void | CH₂ |
| 10 | Ph | Ph | F, ethyl, NMe₂, | 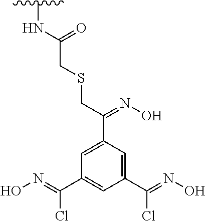 [SMILES: *NC(=O)CSC/C(=N/O)c1cc(/C(Cl)=N/O)cc(/C(Cl)=N/O)c1] | void | void | CH₂ |
| 11 | Ph | Ph | F, ethyl, NMe₂, | 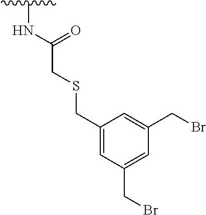 [SMILES: *NC(=O)CSCc1cc(CBr)cc(CBr)c1] | void | void | CH₂ |
| 12 | Ph | Ph | F, ethyl, NMe₂, | 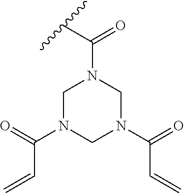 [SMILES: *C(=O)N1CN(C(=O)C=C)CN(C(=O)C=C)C1] | void | void | CH₂ |
| 13 | Ph | Ph | F, ethyl, NMe₂, | 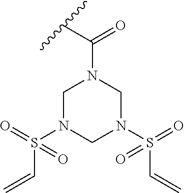 [SMILES: *C(=O)N1CN(S(=O)(=O)C=C)CN(S(=O)(=O)C=C)C1] | void | void | CH₂ |

The compounds of Formula I can be attached to via a linker or incorporated into another agent. Nonlimiting examples include the following.

TABLE 2
Agents modified with the compounds of Formula I

| | |
|---|---|
| Original agent A | 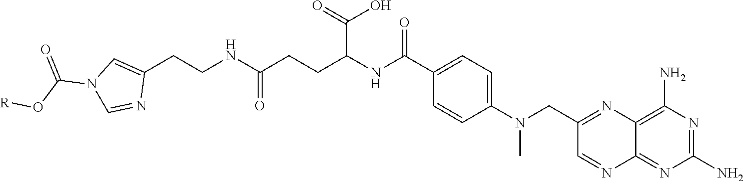 |
| Agent modified with a com- ound of Formula I | 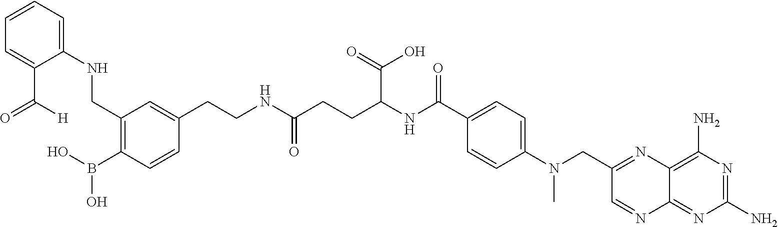 [SMILES: CN(Cc1cnc2nc(N)nc(N)c2n1)c1ccc(C(=O)NC(CCC(=O)NCCc2ccc(B(O)O)c(CNc3ccccc3C=O)c2)C(=O)O)cc1] |
| Original agent B | 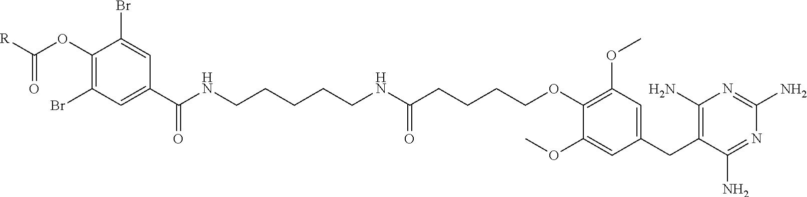 |
| Agent modified with a com- ound of Formula I | 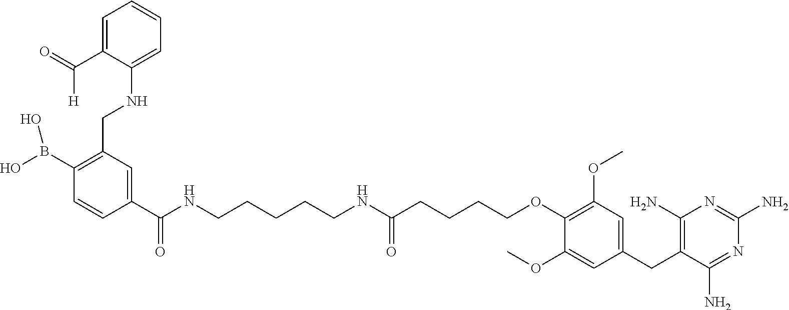 [SMILES: COc1cc(Cc2c(N)nc(N)nc2N)cc(OC)c1OCCCCC(=O)NCCCCCNC(=O)c1ccc(B(O)O)c(CNc2ccccc2C=O)c1] |

The above modified agents can be readily prepared via chemistry techniques known in the art. The following routes illustrate the modification approach to the agents.

### Modification of Agent A

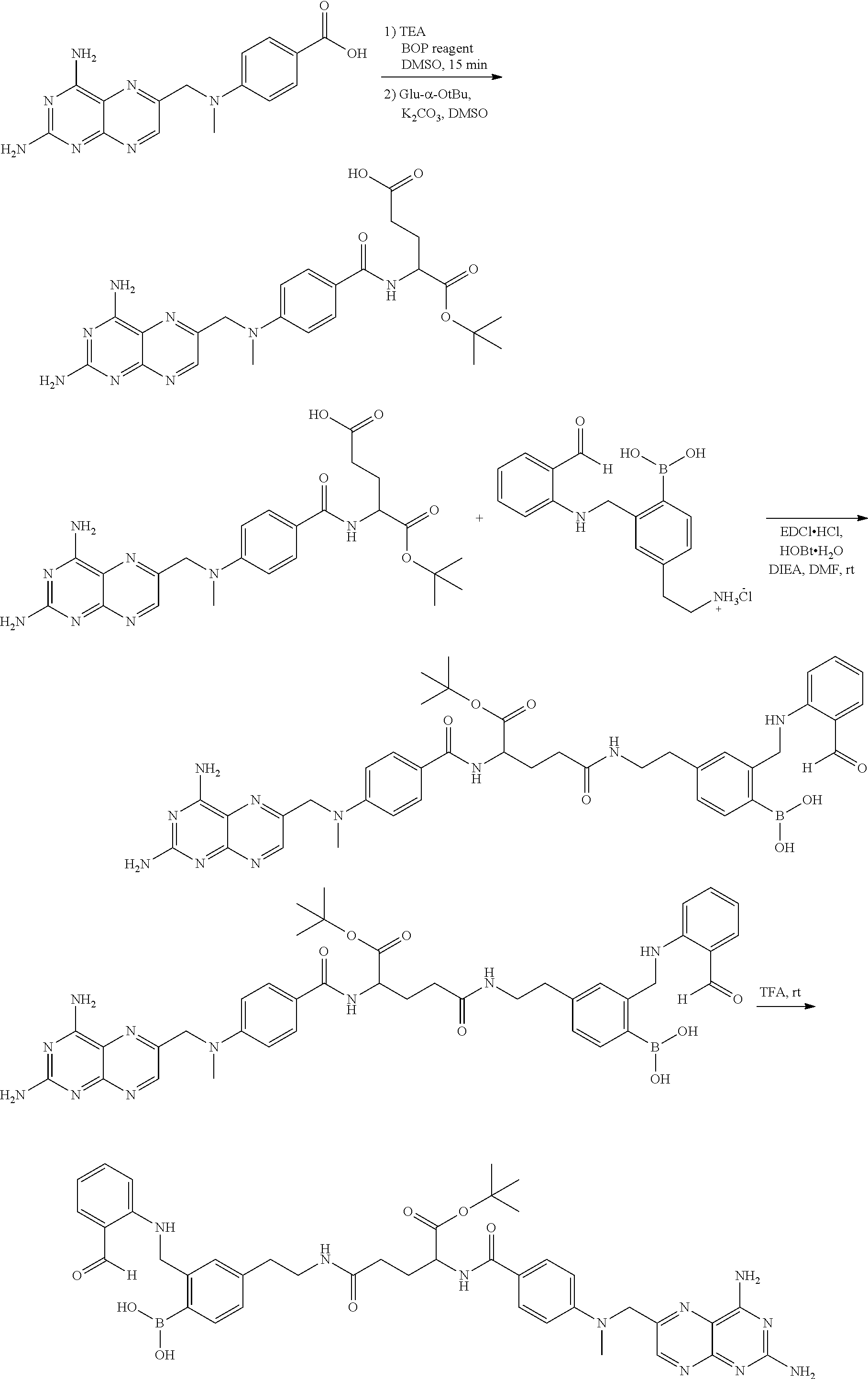

### Modification of Agent B

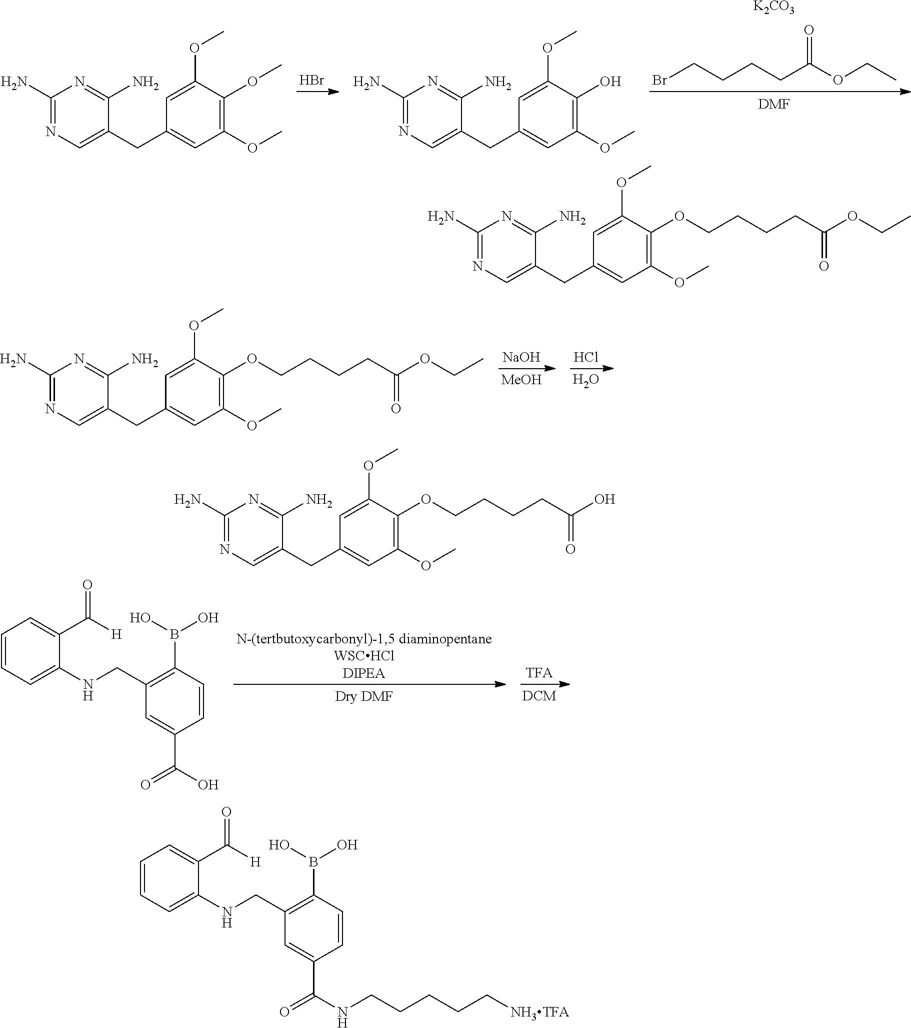

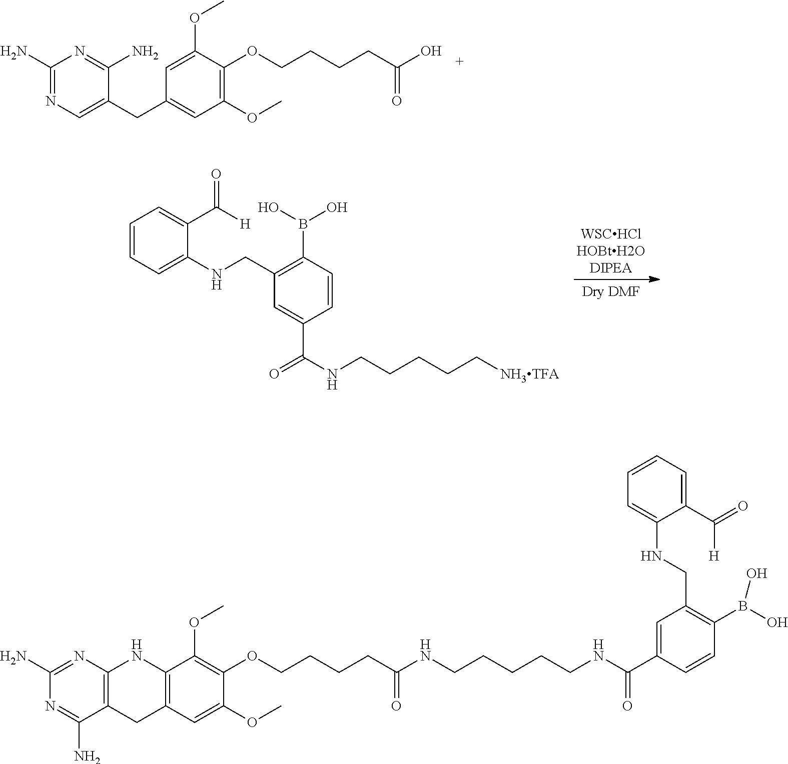

Another aspect provides a phage display library comprising phage particles comprising a peptide disclosed herein. General procedures for preparing a phage display library is known in the art, including those disclosed in U.S. Pat. No. 11,655,468, the entire disclosure of which is hereby incorporated by reference.

Another aspect of the present disclosure provides a pharmaceutical composition containing a therapeutically effective amount of the compound disclosed herein and a pharmaceutically acceptable carrier.

The pharmaceutical composition may also contain one or more physiologically acceptable surface-active agents, additional carriers, diluents, excipients, smoothing agents, suspension agents, film forming substances, and coating assistants, or a combination thereof, and a composition disclosed herein. Acceptable additional carriers or diluents for therapeutic use are well known in the pharmaceutical art, and are described, for example, in Remington's Pharmaceutical Sciences, 18th Ed., Mack Publishing Co., Easton, PA (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid, and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, microcrystalline cellulose, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The pharmaceutical compounds described herein can be administered to a human patient per se, or in pharmaceutical compositions where they are mixed with other active ingredient(s), as in combination therapy, or suitable carriers or excipient(s). In some embodiments, a dosage form includes those forms in which the compound is administered per se. In addition, a dosage form may include a pharmaceutical composition. In any case, the dosage form may comprise a sufficient amount of the compound to treat a disease as part of a particular administration protocol, as would be understood by those of skill in the art. Techniques for formulation and administration of the compounds of the instant application may be found in “Remington's Pharmaceutical Sciences,” Mack Publishing Co., Easton, PA, 18th edition, 1990.

The pharmaceutical compositions may be manufactured in a manner that is itself known, e.g., by means of conventional mixing, dissolving, granulating, dragee-making, levigating, emulsifying, encapsulating, entrapping or tabletting processes.

Pharmaceutical compositions may be formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, diluents, carriers, and excipients may be used as suitable and as understood in the art; e.g., in Remington's Pharmaceutical Sciences, above.

Another aspect provides a method of screening a peptide against a target of interest. In some embodiments, the peptide is attached to a bacteriophage coat protein. The method includes:
a) screening the peptide disclosed herein with the target of interest;
b) identifying the peptide as a high affinity peptide when its affinity to the target is determined to be submicromolar.
c) Conjugating the selected peptide binders with an agent.

Another aspect provides a method of developing a novel therapeutic agent against a target of interest. The method includes conjugating the identified peptide disclosed herein with a therapeutic agent against the target. In some embodiments, the target is a bacteria of Staphylococcus aureus or Acinetobacter baumannii strain.

### EXAMPLES

### Example 1

The reversibility of the diazaborine conjugation was investigated via a dilution experiment. A 10 mM mixture of RMR1 and lysine was incubated overnight, and then diluted 20 times. The ¹H-NMR data show that the RMR1-lysine conjugate dropped from 50% to 13% over time, confirming the autonomous reversibility of the conjugation. The thermodynamic and kinetic parameters of RMR1-amine conjugation was determined the via a set of UV-vis experiments. First, the apparent K_{d} values was measured for RMR1 binding lysine, MEA and a glycine amide respectively. All three amines gave a comparable K_{d} around 1 mM. The dissociation rate of the diazaborines was determined by diluting a 10 mM solution to 50 μM and monitoring the relaxation kinetics using UV-vis spectroscopy. As the diluted concentration is much below the K_{d} values, the relaxation kinetics should be dictated by the dissociation rate. Exponential curve fitting gives comparable dissociation rate constants (k₋₁) for the three amines with a value of 2.6×10⁻⁵ s⁻¹ for lysine. The slow dissociation of the diazaborines was further corroborated by ¹H-NMR analysis of diluted samples.

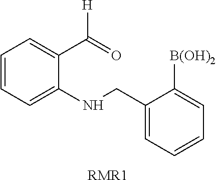

With the K_{d} and k₋₁ values, the forward reaction rate of the RMR1-lysine conjugation to be 2.1×10⁻² M⁻¹s⁻¹ was estimated. In comparison to the iminoboronate formation, which shows instantaneous equilibrium, the RMR1-lysine conjugation exhibits slower kinetics but more favorable thermodynamic profile. This is confirmed by a competition experiment, in which the iminoboronate conjugates of lysine slowly exchanged with RMR1 to give the diazaborine as the predominant end product.

The distinct kinetic profiles of the two reversible lysine conjugations present an opportunity to compare the fast and slow dissociating warheads towards developing lysine-targeted reversible covalent inhibitors. Towards this end, an important enzyme of Staphylococcus aureus, sortase A (SrtA) was used as a model system. SrtA is an appealing anti-Staph target as SrtA inhibition is not bactericidal and hence does not force the acquisition of resistance mechanisms. Through phage display, a cyclic peptide W7 was identified that binds SrtA with low micromolar potency. Computational docking indicates that W7 binds to the SrtA active site with the N-terminus and C-terminus projected away from the protein and potentially suitable for warhead incorporation.

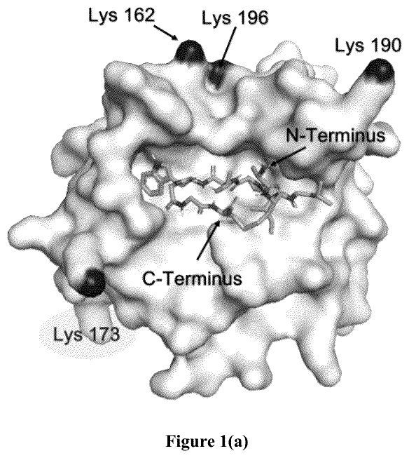

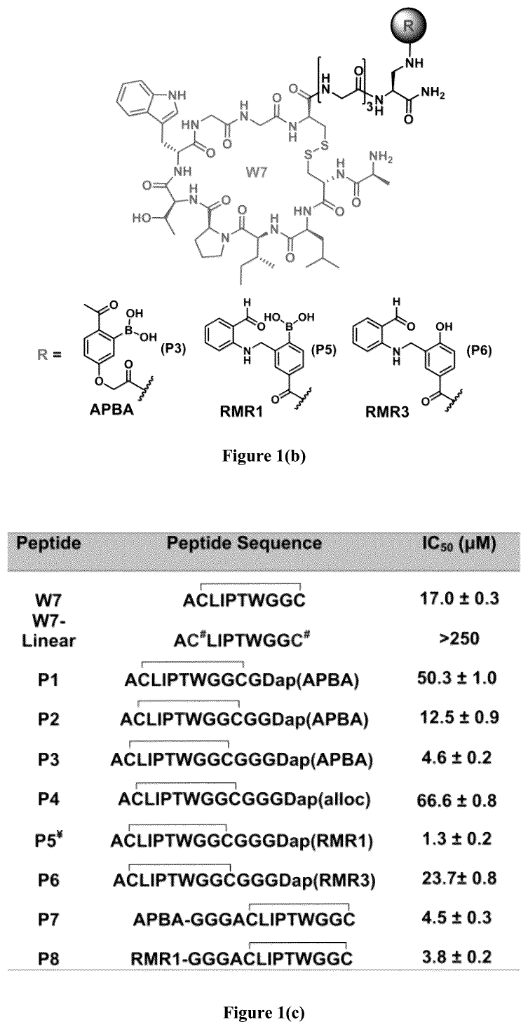

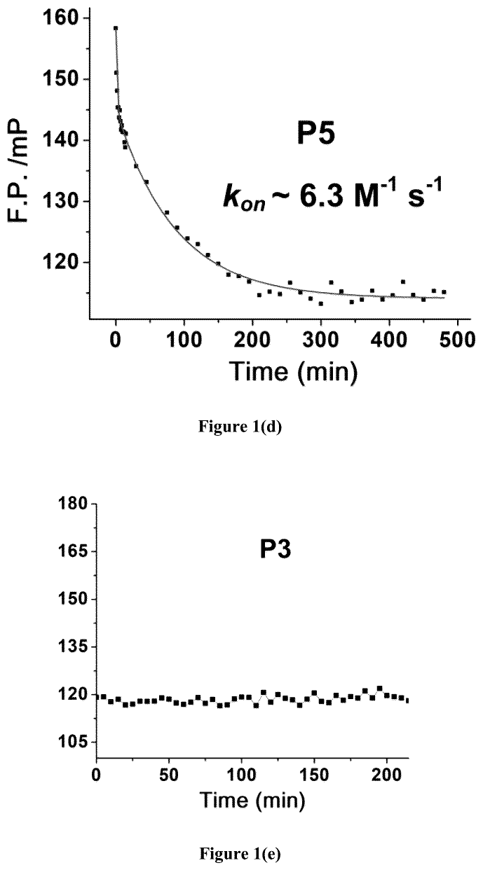

For peptide conjugation, a —COOH derivative of RMR1 and 2-APBA was synthesized respectively. These —COOH derivatives were either conjugated directly to the W7 N-terminus or to the C-terminus via an orthogonally protected diaminopropionic acid (Dap) residue (FIG. 1). The resulting peptides were assessed for SrtA binding through a fluorescence polarization-based competition assay, in which a fluorophore labeled W7 (W7-F) was used as a reporter. Fitting the titration curves yielded the IC₅₀ values, which can be approximated as K_{d}'s as the reporter peptide was used at much lower concentrations (0.2 μM). Interestingly, RMR1 installed on the C-terminus was found to undergo slow intramolecular conjugation with the N-terminal amine, and hence, the peptide P5 was N-acetylated to avoid cyclization. Comparative studies of W7 and P3 shows that N-acetylation has little effect on their SrtA binding potencies.

The unmodified W7 (no warhead) gave an IC₅₀ of 17 μM. Installing Dap(APBA) onto the C-terminus with a single glycine spacer (P1) diminished the SrtA-binding potency to 50 μM. Interestingly, extending the spacer length regained the potency, yielding an IC₅₀ of 12.5 and 4.6 μM for P2 and P3 respectively. The control peptide P4 afforded a 15-fold higher IC₅₀ than P3, highlighting the importance of the APBA warhead. Replacing the APBA warhead with RMR1 (P5) gave even greater potency with an IC₅₀ of 1.3 μM. Remarkably, P5 is 18 times more potent than the control peptide P6, which incorporates RMR3 as a non-reactive RMR1 analogue. Conjugating APBA or RMR1 onto the N-terminus of W7 significantly improves the peptide's binding to SrtA as well, yielding an IC₅₀ of 4.5 and 3.8 μM for P7 and P8 respectively. Finally, the postulated covalent binding of P5 to SrtA was confirmed by LC-MS. To pinpoint the site of conjugation, peptide mapping experiments was performed, in which the SrtA-P5 conjugate was subjected to trypsin digestion and then LC-MS analysis. The results allowed identification of K173 as the conjugation site for P5, consistent with the fact that K173 exhibits the shortest distance to the peptide's C-terminus as seen in the results of the docking studies.

Kinetic characterization of the P5-SrtA binding yielded a kₒₙ of 6.3 M⁻¹ s⁻¹, which is ˜300 times faster than the RMR1-lysine conjugation. The k_{off} was determined to be 8.2×10⁻⁶ s⁻¹, 4 times slower than the RMR1-lysine conjugation. The faster on-rate and slower off-rate are expected for the cooperative action between the covalent warhead and the W7 peptide that binds SrtA noncovalently. In contrast to P5, the P3-SrtA binding reached equilibrium instantaneously, consistent with the rapid kinetics of iminoboronate formation.

The peptides for SrtA inhibition on live S. aureus cells was further tested. A fluorescently labeled SrtA substrate (FAM-GSLPETGGS) was mixed with S. aureus in LB media with or without a peptide inhibitor. The SrtA-mediated bacterial labeling was assessed using fluorescence microscopy and flow cytometry. Without an inhibitor, the bacterial cells exhibited strong fluorescence staining after incubation with the SrtA substrate. The peptide inhibitors inhibited SrtA-mediated fluorescence labeling of the cells in a concentration-dependent manner. Curve fitting yielded an IC₅₀ of 10.7 μM for W7, which is on par with the IC₅₀ determined from enzyme binding studies. Also consistent with the earlier results, both P3 and P5 exhibited higher potency of inhibition, yielding IC₅₀ of 3.7 and 2.9 μM respectively.

Although a similar IC₅₀ was observed for P3 and P5 in our SrtA inhibition assay, it was reasoned that the slow dissociation of P5 could afford kinetic benefit, which manifests as longer residence time in the enzyme leading to long-lasting inhibition. To test this hypothesis, the S. aureus cells was treated with P5 and then the unbound inhibitor was washed away before the fluorescent SrtA substrate was added. The S. aureus cells were subjected to flow cytometry analysis after 6 hours of incubation. The results show that, even with washing, P5 efficiently inhibited the SrtA-mediated fluorescence labeling of S. aureus. In sharp contrast, W7 and P3 only elicited marginal reduction of the fluorescence of the cells. These results nicely demonstrate the long-lasting SrtA inhibition by P5, which presumably results from its slow dissociation kinetics.

### Example 2

The following scheme illustrates the synthesis of an example compound. The synthesis can be readily modified for the preparation of other compounds under Formula I. The intermediates in the scheme are either commercially available or can be prepared using routine chemistry. For example, intermediate 5 can be constructed via an alkylation reaction between an aryl amine (left moiety) with a substituted bromo-benzyl (right moiety).

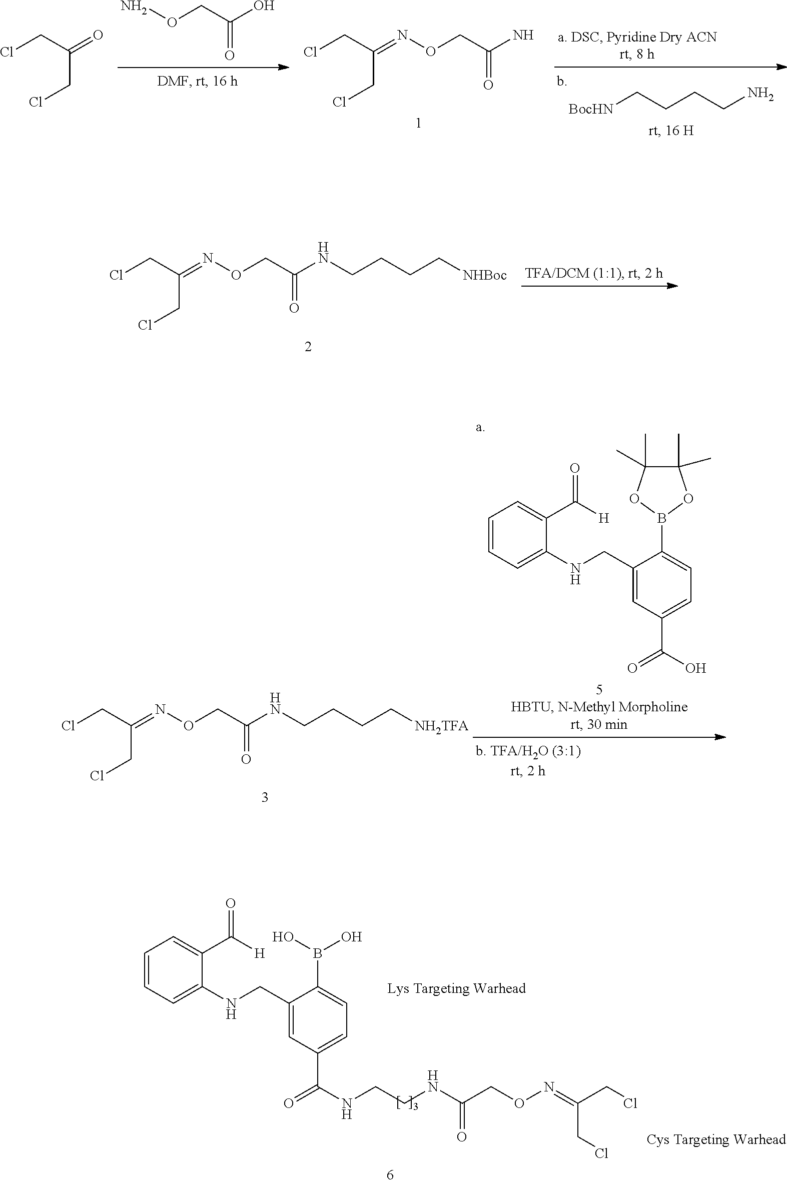

All references cited herein are incorporated herein by reference in their entireties. It will be appreciated by persons skilled in the art that the present invention is not limited to what has been particularly shown and described. Rather, the scope of the present invention is defined by the claims which follow. It should further be understood that the above description is only representative of illustrative examples of embodiments. The description has not attempted to exhaustively enumerate all possible variations. The alternate embodiments may not have been presented for a specific portion of the invention, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

We claim:

1. A compound of Formula I, 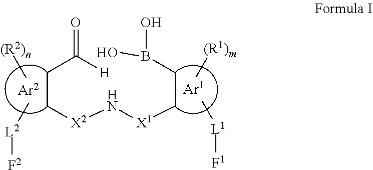
wherein Ar¹ and Ar² are each independently a 5-10 membered aromatic ring,
X¹ is C₁₋₂ alkylene or CH═CH (optionally substituted),
X² is void or CH₂,
L¹ and L² are each independently void or a linker, provided that L¹ and L² are not void at the same time,
F¹ and F² are each independently void or a functional group capable of reacting with OH, SH or amino group to form a covalent bond,
R¹ and R² in each instance are independently selected from the group consisting of C₁₋₆alkyl, C₁₋₄alkyl-C(O), CN, NO₂, OH, halogen, C₁₋₄alkyl-O, N(R^{a})₂, C₁₋₆alkyl-O—C(O), C₁₋₆alkyl-C(O)—O, C₁₋₆ alkyl-C(O)—NR^{a}, C₁₋₆alkyl-NR^{a}—C(O), wherein R^{a} in each instance is independently H or C₁₋₆alkyl,
m and n are each independently 0, 1, 2, 3, 4 or 5.

2. The compound of claim 1, wherein Ar¹ and Ar² are each independently an optionally substituted phenyl or an optionally substituted 5-10 membered heteroaryl.

3. The compound of claim 1, wherein Ar¹ and Ar² are each independently selected from the group consisting of 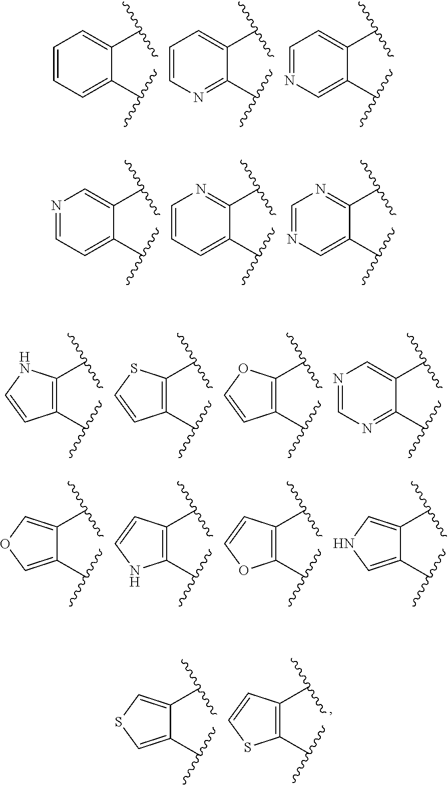
wherein each of the Ar¹ and Ar² are optionally substituted.

4. The compound of claim 1, wherein one of Ar¹ and Ar² is an optionally substituted phenyl and the other is an optionally substituted 5-10 membered heteroaryl.

5. The compound of claim 1, wherein both of Ar¹ and Ar² are each an optionally substituted phenyl.

6. The compound of claim 1, wherein X¹ is methylene.

7. The compound of claim 1, wherein X¹ is ethylene or ethenylene.

8. The compound of claim 1, wherein X² is void.

9. The compound of claim 1, wherein one or both of L¹ and L² independently comprise one or more units selected from the group consisting of —C(O)NHC₂₋₈alkyleneNH—, —NHC₂₋₈alkyleneNH—, —NHC(O)C₁₋₆alkyleneNH—, —NHC(O)NHC₁₋₈alkyleneNH—, C₁₋₈alkylene, NH—C₁₋₈alkylene, amino acid, heterocyclic, —C₁₋₈alkyleneC(O)NR^{a}C₁₋₈alkylene-, —(CH₂)ₐO(CH₂CH₂O)_{c}—, —C₁₋₈alkyleneC(O)—, and —S—S—, wherein a, b, and c are each an integer selected from 0 to 25, all subunits included.

10. The compound of claim 1, wherein one or both of L¹ and L² independently comprise one or more units selected from the group consisting of —C(O)NHC₂₋₆alkyleneNH—, —NHC₂₋₈alkyleneNH—, —NHC(O)C₁₋₆alkyleneNH—, —NHC(O)NHC₁₋₆alkyleneNH—, C₁₋₈alkylene, and NH—C₁₆alkylene,

11. The compound of claim 1, wherein F¹ and F², when present, are independently selected from the group consisting of COOH, maleimide, 2′-pyridyldithio, vinyl sulfone, bromo or iodo acetamide, azide, alkyne, dibenzocyclooctyl (DBCO), carbonyl,

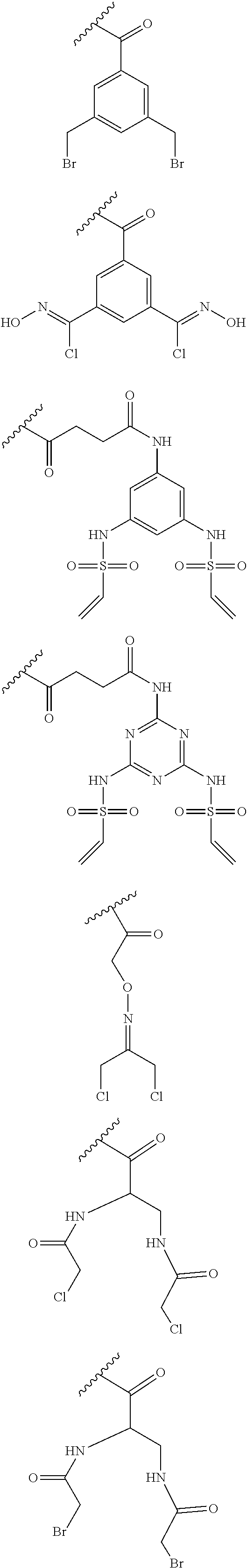

12. The compound of claim 1, wherein at least one of F¹ and F² is COOH.

13. The compound of claim 1, wherein the compound is selected from the group consisting of 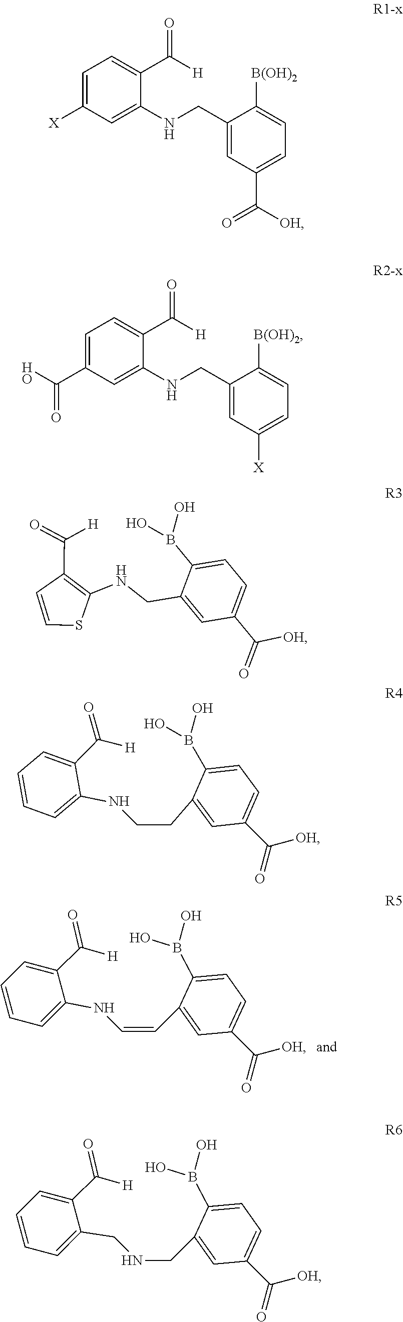
wherein X is —NO₂, —OMe, or —NMe₂.

14. A peptide comprising a residue modified by the compound of claim 1, wherein the peptide is represented by Formula II, 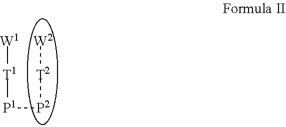
Wherein the circled W²-T²-P² is optional,
Wherein W¹ and W² are each independently derived from Formula I, T¹ and T² are each independently a di-functional or tri-functional linkage, P¹ and P² are each independently a peptide comprising three or more amino acid residues.

15. The peptide of claim 14, wherein T¹ is a bifunctional linkage.

16. The peptide of claim 14, wherein T¹ is a trifunctional linkage linking W¹ with two amino acid residues.

17. The peptide of claim 14, wherein W²-T²-P² is present and wherein T¹ and T² are linked to each other.

18. The peptide of claim 14, wherein the modified residue is a cysteine residue or a lysine residue.

19. The peptide of claim 14, comprising two modified cysteine residues resulting from two cysteines reacting with one of F¹ and F².

20. A phage display library comprising phage particles comprising a peptide of claim 14.
